(19) United States
(12) Patent Application Publication
(10) Pub. No.: US 2025/0360018 A1
(43) Pub. Date: Nov. 27, 2025

(54) THERAPEUTIC DEVICE

(71) Applicant: SHEINER ENGINEERING & DESIGN, LLC, Whitestone, NY (US)

(72) Inventors: Brandon Michael SHEINER, Whitestone, NY (US); Semyon RODKIN, Plainview, NY (US)

(21) Appl. No.: 18/940,408

(22) Filed: Nov. 7, 2024

Related U.S. Application Data
(60) Provisional application No. 63/597,311, filed on Nov. 8, 2023.

(51) Int. Cl. A61F 5/10; A61F 5/01; A61F 5/05

(52) U.S. Cl. CPC A61F 5/10; A61F 5/0118; A61F 5/05

(57) ABSTRACT

Provided herein are therapeutic devices for the manipulative treatment of soft tissue of the human body. The devices of the present disclosure comprise a brace comprising a curved body that partially or completely encompasses a portion of an arm, and a digit immobilizer comprising at least one plate to receive a hand in a flat position. The brace and the digit immobilizer are in communication with one another through at least one strap extending from the curved body of the brace to one of the plates of the digit immobilizer. Related methods of use and kits are further provided.

### CROSS-REFERENCE TO RELATED APPLICATIONS

This application claims priority to and the benefit of U.S. Provisional Application Ser. No. 63/597,311, which was filed on Nov. 8, 2023, the contents of which is hereby incorporated by reference in its entirety.

### BACKGROUND

Disorders to the elbow, arms, forearms, and hands, commonly referred to as upper extremity musculoskeletal disorders (hereinafter “UEMSDs”), are disabling injuries that impact the lives of many. Such UEMSDs are injuries or pain in the human musculoskeletal system, including the joints, ligaments, muscles, nerves, tendons, and structures that support the arms and hands. UEMSDs can arise from a variety of causes, including intense exertion, repetitive strain, or from repeated exposure to force, vibration, or improper posture. Common examples of UEMSDs include carpal tunnel syndrome, tendonitis, epicondylitis (i.e., tennis elbow), and hand-arm vibration syndrome.

In today's world, there is a growing trend toward healthy lifestyles. Consequently, exercise in many forms has become common. But with exercise comes an increased risk of injury. Many sports and forms of exercise focus on the use of repetitive motions for various purposes, such as to build strength or to achieve athletic goals. One of the more common injuries to individuals engaging in such repetitive motions is lateral epicondylitis, commonly known as tennis elbow. Individuals experiencing lateral epicondylitis suffer from inflammation and micro-tearing of the tendons that join the forearm muscles on the outside of the elbow, which causes pain and tenderness at the site of injury. Lateral epicondylitis is largely associated with overuse of the muscles and tendons, usually from repetitive motions, but the elderly and people who work trades requiring constant use of tools are likewise at a similar risk. Mild cases of lateral epicondylitis may be treated through simple rest and use of anti-inflammatory measures, such as medication or icing the injured area. However, moderate to severe cases usually require therapeutic motions and exercises.

In addition to tennis elbow, there are many other injuries and surgical procedures (post-surgery) which require an affected individual to rehabilitate their forearm, hand, elbow, and associated muscles and tendons within the arm.

Common therapeutic exercises for such injuries include stretching and strengthening of the lateral and medial muscle groups of the forearm. For example, engaging in these stretches typically requires a user to manually position the wrist of the affected arm to the flexed or extended position by placing pressure on it with the other hand to assist in stretching the weakened tissue. Similarly, engaging in these strengthening exercises requires a user to be stationary in order to rest the arm on a flat surface and use a means of resistance, such as a dumbbell weight, to exercise the targeted muscles.

These therapeutic motions, while effective, can become inconvenient and disruptive to the individual performing them since the proper form for these exercises requires use of both of hands and/or for the user to be stationary for the duration of the exercise. While products exist that allow a user to strengthen their forearm muscles without remaining stationary and there are similarly some products on the market which allow a user to stretch soft tissue in the forearms and wrists without the continued use of a second hand, there are no solutions on the market which allow a user to both stretch and strengthen the forearm muscles in a manner that does not necessitate being stationary or use of an off-hand for the duration of the exercise. Furthermore, given that these strengthening and stretching exercises are often performed in sequence with one another, it would be sensible for a single product solution to allow for both strengthening and stretching of a user's muscles with limited adjustments to the device being necessary, while also freeing the use of the other hand for non-therapeutic purposes.

It is further necessary for such a product to be configured in such a way as to immobilize the digits of the wrist being stretched, which would otherwise result in improper stretching of the targeted muscles. There are no solutions on the market that solves each of these problems.

There is thus a need for a versatile, comprehensive, portable, storable, and efficient devices to aid users suffering from an upper extremity musculoskeletal disorder with stretching and strengthening the forearm muscles while allowing the user to move around, perform other tasks with the hand not being rehabilitated during the exercise, and allow a user to conveniently switch between strengthening and stretching exercises. There is further need for such a product solution to facilitate orientation of the hand and digits according to proper stretching techniques through immobilization of the digits.

### SUMMARY

Accordingly, provided herein are versatile, comprehensive, portable, storable, and efficient devices to aid users suffering from an upper extremity musculoskeletal disorder with stretching and strengthening the forearm muscles while allowing the user to move around, perform other tasks with the hand not being rehabilitated during the exercise, and allow a user to conveniently switch between strengthening and stretching exercises. The devices of the present disclosure provide a product solution to facilitate orientation of the hand and digits according to proper stretching techniques through immobilization of the digits.

The device of the present disclosure utilizes a modular design comprising of one or more braces positionable on an arm, and one or more plates for immobilizing a user's hand, each in communication with one another through the use of connecting members such as straps and tethers. The plates may be tightened sufficiently around a user's hand to prevent unintended bending of the fingers or movement of the hand which may otherwise result in improper stretching of the intended forearm and wrist muscles. The brace(s) may additionally be tightened around a user's arm to remain stationary in place. The hand plates are connectable to a brace positioned on a forearm or around a user's bicep through the use of a strap, which may be used for stretching or strengthening motions. Further optional tethers are connectable with the brace with an upper arm support brace. The upper arm support is optional but may be advantageous to help prevent slippage of the brace towards the hand if placed on the forearm. The brace may comprise a cushion member to add comfort for use without the upper arm support, as well as security against slippage during use.

During use, a user puts the device onto an arm in need of therapy or rehabilitation. The hand is inserted between the plates, which are tightened through the use of bands to immobilize the user's hand and digits. The brace is placed around a portion of the arm (e.g., the forearm or the bicep) and is tightened to prevent slippage during usage. If using the upper arm support, the user places the elbow/upper arm brace on their upper arm around or above the elbow and similarly tightens to fit snugly around the arm; the tethers connecting the brace to the upper arm support must be adjusted to fit the user's arm length. The user then adjusts the length of the straps which connect the hand plates to the brace for each respective exercise type. For example, to stretch the lateral group of forearm muscles, the strap attached to the lateral plate (i.e., the plate abutting the back of the hand) is loosened, while the strap attached to the medial plate (i.e., the plate abutting the palm of the hand) is tightened. Inversely, to stretch the medial group of forearm muscles, the strap attached to the medial plate is loosened and the strap attached to the lateral plate is tightened. In strengthening mode, an alternate set of straps having an interchangeable elastic resistance member are used for connecting the plates to the brace. These straps allow for elastic resistance to be applied while the user moves their hand in a range of motion similarly to wrist eccentric and concentric curls.

In a first aspect, the present disclosure is directed to a therapeutic device [Device 1] for the manipulative treatment of soft tissue of the human body, the device comprising:
a brace comprising a curved body that partially or completely encompasses a portion of an arm; and
a digit immobilizer comprising at least one plate to receive a hand in a flat position,
   wherein the brace and the digit immobilizer are in communication with one another through at least one strap extending from the curved body of the brace to one of the plates of the digit immobilizer.

In further embodiments, Device 1 is further defined as follows.
1.1 Device 1, wherein the curved body of the brace comprises a gap at opposing ends of the curved body.
1.2 Device 1 or 1.1, wherein the brace comprises at least one fastener spanning the gap to secure the brace firmly in place on a user's arm.
1.3 The preceding Device, wherein the fastener is adjustable to constrict or expand the gap.
1.4 Device 1.3 or 1.4, wherein the fastener is adjustable to restrict or expand the radius of the curved body.
1.5 Any of the preceding Devices, wherein the brace comprises two or more fasteners (e.g., two fasteners).
1.6 Any of Devices 1.3-1.6, wherein the fasteners comprise sufficient elastic character to enclose the opening for a tight fit around a user's arm.
1.7 Any of the preceding Devices, wherein the brace is sized to encompass a user's forearm partially or completely.
1.8 Any of Devices 1.1-1.6, wherein the brace encompasses a user's arm proximal to the elbow, e.g., a portion of the arm surrounding the user's bicep.
1.9 Device 1, wherein the curved body of the brace comprises a C-shaped cross-section.
1.10 Any of the preceding Devices, wherein the curved body of the brace is composed of a pliable material (e.g., plastic) or an elastic material.
1.11 Any of the preceding Devices, wherein the brace comprises a cushion member at one or both of the proximal and distal ends of the brace.
1.12 Any of the preceding Devices, wherein the brace comprises a cushion member at the distal end of the brace.
1.13 The preceding Device, wherein the cushion member is made from a shape-retaining material (e.g., plush, memory foam or silicone material).
1.14 The two preceding Devices, wherein the cushion member is coated in a grip enhancing material.
1.15 Any of the preceding Devices, wherein the at least one strap extends from a distal end of the curved body of the brace to one of the plates of the digit immobilizer.
1.16 Any of the preceding Devices, comprising a pair of straps each extending respectively from a distal end of the curved body of the brace to one of the plates of the digit immobilizer.
1.17 The preceding Device, wherein the first strap extends from a distal end of the curved body on a lateral-facing portion of the brace to a lateral plate, and the second strap extends from a distal end of the curved body on a medial-facing portion of the brace to a medial plate.
1.18 Any of the preceding Devices, wherein the at least one strap is either a soft tissue-stretching strap or a strengthening strap.
1.19 The preceding Device, wherein the strap is a soft tissue-stretching strap having an adjustable length.
1.20 The preceding Device, wherein the soft tissue-stretching strap is substantially or completely non-elastic.
1.21 The preceding Device, wherein the soft tissue-stretching strap is non-elastic.
1.22 Any of Devices 1.18-1.21, wherein the soft tissue-stretching strap has a fixable length such that when in place extending between the brace and the digit immobilizer, one of extension or flexion of the wrist is limited.
1.23 Device 1.18, wherein the strap is a strengthening strap comprising an elastic resistance member.
1.24 The preceding Device, wherein the strengthening strap comprises an elastic resistance member having an elastic resistance such that when the strengthening strap is in place extending between the brace and the digit immobilizer, resistance force is placed on the wrist against one of extension or flexion.
1.25 The preceding Device, wherein the strengthening strap may be stretched through motion of the wrist opposing the resistance force (i.e., such that the lateral muscles (i.e., those responsible for extension) or the medial muscles (i.e., those responsible for flexion) are strengthened through resistance training).
1.26 Any of Devices 1.18 or 1.23-1.25, wherein the strengthening strap comprises an elastic resistance member engaged with and running between opposite tethers.
1.27 Any of Devices 1.18 or 1.23-1.26, wherein the strengthening strap comprises an elastic resistance member engaged with and running between opposite tethers, such that when the strengthening strap is in place extending between the brace and the digit immobilizer, resistance force is placed on the wrist against one of extension or flexion.
1.28 The preceding Device, wherein the strengthening strap may be stretched through motion of the wrist opposing the resistance force (i.e., such that the lateral muscles (i.e., those responsible for extension) or the medial muscles (i.e., those responsible for flexion) are strengthened through resistance training).
1.29 Any of the preceding Devices, wherein the digit immobilizer prevents articulation of the joints in the fingers.
1.30 Any of the preceding Devices, wherein the digit immobilizer comprises two plates.
1.31 Any of the preceding Devices, wherein the digit immobilizer comprises a medial plate disposable with or adjacent to the palm of a hand and a lateral plate disposable with or adjacent to the back of the hand.
1.32 Any of the preceding Devices, wherein the plates of the digit immobilizer are each substantially planar and comprise inner surface (i.e., surface contactable against the skin) and an outer surface (i.e., surface opposite the inner surface).
1.33 Any of the preceding Devices, wherein the plates of the digit immobilizer are each defined by a square, rectangular, rounded, or irregular perimeter.
1.34 Any of the preceding Devices, wherein the plates of the digit immobilizer are each defined by a rectangular perimeter.
1.35 Any of the preceding Devices, wherein the plates of the digit immobilizer each generally conform to the dimensions of a human hand.
1.36 Any of the preceding Devices, wherein the plates of the digit immobilizer comprise a plurality of grooves along the perimeter to receive at least one band (e.g., one band or two bands).
1.37 The preceding Device, wherein the grooves are configured to accommodate bands that apply pressure between the plates against the hand so as to keep the plates disposed firmly in place.
1.38 Device 1.36 or 1.37, wherein the bands are adjustable or elastic.
1.39 Any of Devices 1.36-1.38, wherein the bands are comprised of an elastic material and have sufficient elastic resistance to prevent articulation of the digits when received within the grooves of the medial and lateral plates.
1.40 Any of the preceding Devices, wherein the outer surface of each of the medial and lateral plates comprises an engagement mechanism to provide an attachment point for the at least one strap.
1.41 Any of the preceding Devices, wherein the digit immobilizer comprises a glove in which the plate(s) may be received to fit directly to a user's hand.
1.42 Any of the preceding Devices, wherein the digit immobilizer comprises a glove which medially receives a first plate to position against the palm, and laterally receives a second plate to position against the back of the hand.
1.43 Any of the preceding Devices, further comprising an upper arm support having a curved body.
1.44 The preceding Device, wherein the upper arm support is angled to accommodate a neutral positioning of the elbow.
1.45 Device 1.43-1.44, wherein the upper arm support is sized to partially or completely encompass a portion of a user's arm.
1.46 Any of Devices 1.43-1.45, wherein the upper arm support is sized to partially or completely encompass a portion of a user's upper forearm and optionally the user's elbow.
1.47 Any of Devices 1.43-1.46, wherein the upper arm support comprises a body having a C-shaped cross-section.
1.48 Any of Devices 1.43-1.47, wherein the curved body of the upper arm support comprises a gap at opposing ends of the curved body.
1.49 Any of Devices 1.43-1.48, wherein the upper arm support comprises at least one fastener spanning the gap to secure the brace firmly in place on a user's arm.
1.50 Any of Devices 1.43-1.49, wherein the fastener is adjustable to constrict or expand the gap.
1.51 Any of Devices 1.43-1.50, wherein the upper arm support comprises two fasteners.
1.52 Any of Devices 1.43-1.51, comprise sufficient elastic character to enclose the opening for a tight fit around a user's arm.
1.53 Any of Devices 1.43-1.52, further comprising at least one connecting member to secure a distal end of the upper arm support to a proximal end of the brace.
1.54 Any of Devices 1.43-1.53, further comprising a pair of oppositely positioned connecting members to secure a distal end of the upper arm support to a proximal end of the brace.
1.55 Any of Devices 1.43-1.54, wherein the curved body of the upper arm support is composed of a pliable material or an elastic material.
1.56 Any of Devices 1.43-1.55, wherein the upper arm support comprises a cushion member at one or both of a proximal or distal end.
1.57 Any of Devices 1.43-1.56, wherein the upper arm support comprises a cushion member at a distal end.
1.58 Any of Devices 1.43-1.57, wherein the cushion member is made from a shape-retaining material (e.g., plush, memory foam or silicone material).
1.59 Any of Devices 1.43-1.58, wherein the cushion member is coated in a grip enhancing material.
1.60 Any of the preceding Devices, wherein the device is useful in the treatment of soft tissue in the arm.
1.61 Any of the preceding Devices, wherein the device is useful in the treatment of tendons, ligaments, or muscles in the arm.
1.62 Any of the preceding Devices, wherein the soft tissue has been subjected to tearing, strain, swelling, irritation or injury.
1.63 Any of the preceding Devices, wherein the device is useful in the treatment of an upper extremity musculoskeletal disorders of the arm.
1.64 Any of the preceding Devices, wherein the device is useful in the treatment of carpal tunnel syndrome, tendonitis, epicondylitis (i.e., tennis elbow), and hand-arm vibration syndrome.
1.65 Any of the preceding Devices, wherein the device is useful in the treatment of lateral epicondylitis.

In a second aspect, the present disclosure is directed to a therapeutic method [Method 1] for rehabilitating soft tissue of the human body, the method comprising the following steps:
providing a therapeutic device comprising:
   a brace comprising a curved body that partially or completely encompasses a portion of an arm; and
   a digit immobilizer comprising at least one plate to receive a hand in a flat position,
      wherein the brace and the digit immobilizer are in communication with one another through at least one strap extending from the body of the brace to one of the plates of the digit immobilizer;
placing the digit immobilizer on a user's hand and placing the brace on a portion of the user's arm; and
attaching the at least one strap between the brace and the digit immobilizer for a desired therapeutic effect.

In further embodiments, Method 1 is further defined as follows.
1.1 Method 1, wherein the therapeutic device is a device according to Device 1.1-1.65.
1.2 Any of the preceding Methods, wherein the desired therapeutic effect is stretching or strengthening of soft tissue.
1.3 Any of the preceding Methods wherein the placing step further comprises looping bands around the plates of the digit immobilizer and fasteners on the brace are tightened such that the digit immobilizer is firmly engaged with the user's hand and the brace is firmly engaged with the user's forearm or a portion of the arm proximal to the elbow (e.g., the user's bicep).
1.4 Any of the preceding Methods, wherein attaching the strap between the brace and the digit immobilizer comprises attachment of a first end of the strap to an outer surface of a medial plate and a second end of the strap to a medial engagement loop on the brace; or attachment of a first end of the strap to an outer surface of a lateral plate and a second end of the strap to a lateral engagement loop on the brace.
1.5 Any of the preceding Methods, wherein attaching the at least one strap between the brace and the digit immobilizer comprises attachment of a first end of a first strap to an outer surface of a medial plate and a second end of the first strap to a medial engagement loop on the brace; and attachment of a first end of a second strap to an outer surface of a lateral plate and a second end of the second strap to a lateral engagement loop on the brace.
1.6 Any of the preceding Methods, wherein the desired therapeutic effect is stretching of soft tissue, and the strap is a soft tissue-stretching strap.
1.7 The preceding Method, wherein the strap is a soft tissue-stretching strap which is has fixable length.
1.8 Method 1.6 or 1.7, wherein the soft tissue-stretching strap is substantially or completely non-elastic.
1.9 Any of Methods 1.6-1.8, wherein the soft tissue-stretching strap is non-elastic.
1.10 Any of the preceding Methods, wherein the desired therapeutic effect is stretching of the lateral forearm muscle group of a user's forearm, and wherein attaching the strap between the brace and the digit immobilizer comprises attachment of a first end of the strap to an outer surface of a medial plate and a second end of the strap to a medial engagement loop on the brace.
1.11 The preceding Method, further comprising the step of adjusting the length of the strap to lock the wrist in a forearm extensor stretching position.
1.12 Any of the preceding Methods, wherein the desired therapeutic effect is stretching of a medial forearm muscle group of a user's forearm, and wherein attaching the strap between the brace and the digit immobilizer comprises attachment of a first end of the strap to an outer surface of a lateral plate and a second end of the strap to a lateral engagement loop on the brace.
1.13 The preceding Method, further comprising the step of adjusting the length of the strap to lock the wrist in a forearm flexor stretching position.
1.14 Any of Methods 1 or 1.1-1.5, wherein the desired therapeutic effect is strengthening of muscle, and the strap is a strengthening strap.
1.15 Method 1.14, wherein the strengthening strap comprises an elastic resistance member with sufficient elastic resistance such that when the strengthening strap is in place extending between the brace and the digit immobilizer, resistance force is placed on the wrist against one of extension or flexion.
1.16 Method 1.14-1.15, wherein the strap is a strengthening strap comprising an elastic resistance member.
1.17 Any of Methods 1.14-1.16, wherein the strengthening strap comprises an elastic resistance member engaged with and running between opposite tethers.
1.18 Any of Methods 1.14-1.17, wherein the strengthening strap is stretched through motion of the wrist opposing the resistance force (i.e., such that the lateral forearm muscles (i.e., those responsible for extension) or the medial forearm muscles (i.e., those responsible for flexion) are strengthened through resistance training).
1.19 Any of Methods 1.14-1.18, wherein the desired effect is strengthening of one or more of the medial forearm muscles, and wherein attaching the strap between the brace and the digit immobilizer comprises attachment of a first end of the strap to an outer surface of a lateral plate and a second end of the strap to a lateral engagement loop on the brace.
1.20 The preceding Method, further comprising stretching the strengthening strap through motion of the wrist opposing the resistance force.
1.21 Any of Methods 1.14-1.18, wherein the desired effect is strengthening of one or more of the lateral forearm muscles, and wherein attaching the strap between the brace and the digit immobilizer comprises attachment of a first end of the strap to an outer surface of a medial plate and a second end of the strap to a medial engagement loop on the brace.
1.22 The preceding Method, further comprising stretching the strengthening strap through motion of the wrist opposing the resistance force.
1.23 Any of the preceding Methods, further comprising the step of placing the upper arm support on a portion of the arm proximal to the brace, and further tightening fasteners on the upper arm support such that the upper arm support is firmly engaged with the user's upper forearm and optionally also the elbow.

In a third aspect, the present disclosure is directed to a kit for a therapeutic device [Kit 1] for the manipulative treatment of soft tissue of the human body, the kit comprising:
a brace comprising a curved body that partially or completely encompasses a portion of an arm;
a digit immobilizer comprising at least one plate to receive a hand in a flat position; and
a strap attachable at one end to the curved body of the brace and at the other end to one of the plates of the digit immobilizer.

In further embodiments, Kit 1 is further defined as follows.
1.1 Kit 1, further comprising an upper arm support.
1.2 Kit 1.1, wherein the upper arm support comprises a curved body.
1.3 Any of the preceding Kits, further comprising a plurality of straps attachable at one end of the curved body of the brace and at the other end to one of the plates of the digit immobilizer.
1.4 Kit 1.2, wherein the plurality of straps comprises at least one soft tissue-stretching strap and at least one strengthening strap.
1.5 Kit 1.3, wherein the plurality of straps comprises two soft tissue-stretching straps and a plurality of strengthening straps, each strengthening strap comprising an elastic resistance member of unique elastic resistance.
1.6 Any of the preceding Kits, further comprising a pair of oppositely positioned connecting members to secure a distal end of the upper arm support to a proximal end of the brace.
1.7 Any of the preceding Kits, further comprising at least one band received within grooves of the plates that during use apply inward pressure to keep the plates disposed firmly against a user's hand.
1.8 Any of the preceding Kits, further comprising a carry bag sized to fit the components of the kit.
1.9 The preceding Kit, wherein the carry bag is comprised of a plastic, fabric or leather material.
1.10 Kit 1.7 or 1.8, wherein the carry bag is sealable (e.g., via drawstring, zipper, hook and loop, or snap buttons).

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

Further aspects, features, benefits, and advantages of the embodiments will be apparent with reference to the following description, appended claims and accompanying drawings where:

FIG. 1 illustrates a front perspective view of components of a therapeutic device in accordance with the present disclosure.

FIG. 2 illustrates a front perspective view of a brace component of a therapeutic device in accordance with the present disclosure. The brace is shown individually in a detached configuration from the digit immobilizer, e.g., as shown in FIG. 1.

FIG. 3 illustrates a front perspective view of a brace component of a therapeutic device in accordance with the present disclosure. The brace is shown without fasteners and individually in a detached configuration from the digit immobilizer, e.g., as shown in FIG. 1.

FIG. 4 illustrates a front perspective view of a digit immobilizer component of a therapeutic device in accordance with the present disclosure. The digit immobilizer is shown individually in a detached configuration from the brace, e.g., as shown in FIG. 1.

FIG. 5 illustrates a front perspective view of a single plate of the digit immobilizer component of a therapeutic device in accordance with the present disclosure.

FIG. 6 illustrates a rear perspective view of a single plate of the digit immobilizer component of a therapeutic device in accordance with the present disclosure.

FIG. 7 illustrates a perspective view of a strengthening strap component of a therapeutic device in accordance with the present disclosure.

FIG. 8 illustrates a perspective exploded view of a strengthening strap component of a therapeutic device in accordance with the present disclosure.

FIG. 8A illustrates a front view of a fastener for use with a strap in accordance with the present disclosure.

FIG. 9 illustrates a front perspective view of an upper arm support component of a therapeutic device in accordance with the present disclosure. The upper arm support is shown individually in a detached configuration from the brace and digit immobilizer, e.g., as shown in FIGS. 11 and 12.

FIG. 10 illustrates a front perspective view of an upper arm support component of a therapeutic device in accordance with the present disclosure. The upper arm support is shown without fasteners and individually in a detached configuration from the brace and digit immobilizer, e.g., as shown in FIGS. 11 and 12.

FIG. 11 illustrates a front perspective view of components of an embodiment of a therapeutic device in accordance with the present disclosure.

FIG. 12 illustrates a rear perspective view of components of an embodiment of a therapeutic device as shown in FIG. 11 in accordance with the present disclosure.

FIG. 13 illustrates a front view of an embodiment of a single plate of the digit immobilizer component of a therapeutic device in accordance with the present disclosure.

FIG. 14 illustrates an exploded perspective view of the plate shown in FIG. 13 within a portion of the digit immobilizer according to an embodiment of the present disclosure.

FIG. 15 illustrates a cutaway view of a digit immobilizer according to an embodiment of the present disclosure.

FIG. 16 illustrates a perspective view of immobilizer according to an embodiment of the present disclosure.

FIG. 17 illustrates front perspective view of components of an embodiment of a therapeutic device in accordance with the present disclosure.

It is to be understood that the figures may not be to scale. Further, the relation between objects in a figure may not be to scale and may have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of ordinary rounding techniques.

It is noted that, as used in this specification and the appended claims, the singular forms “a,” “an,” and “the” include plural referents unless expressly and unequivocally limited to one referent. Thus, for example, reference to “a cover” may include one, two, three or more covers.

The term “distal” generally means situated away from the point of attachment. As used herein, distal is a relative term to describe a portion of the arm positioned away from the shoulder or a feature of the devices disclosed herein corresponding to a portion of the arm positioned away from the shoulder. By way of example, the hand is distal to the elbow, and the fingers are distal to the wrist.

The term “proximal” generally means situated toward the point of attachment. As used herein, proximal is a relative term to describe a portion of the arm positioned toward the shoulder or a feature of the devices disclosed herein corresponding to a portion of the arm positioned toward the shoulder. By way of example, the elbow is proximal to the hand, and the wrist is proximal to the fingers.

The term “medial” generally means situated toward the midline of the body. As used herein, medial refers to portions of the arm as well as features of the devices disclosed herein positioned on the inside of the arm while in the standard anatomical position. That is, medial refers to the portions of the arm and hand that face toward the midline of the body while the arm is positioned at rest at the side of the body. By way of example, the palm of the hand and portion of the forearm directly proximal to the palm are medial to the opposite side of the hand.

The term “lateral” generally means situated away from the midline of the body. As used herein, lateral refers to portions of the arm as well as features of the devices disclosed herein positioned on the outside of the arm while in the standard anatomical position. That is, lateral refers to the portions of the arm and hand that face away from the midline of the body while the arm is positioned at rest at the side of the body. By way of example, the back of the hand and portion of the forearm directly proximal to the back of the hand are lateral to the palm of the hand.

The following discussion includes a description of a personal care therapeutic device 10 (hereinafter interchangeably referred to as either “therapeutic device 10” or “device 10”) for rehabilitation, stretching or strengthening soft tissue in the human body. In particular, therapeutic device 10 comprises a design, features and benefits that allow for the rehabilitation, stretching or strengthening of soft tissue in the arm which has been subjected to tearing, strain, swelling, irritation or injury. Alternate embodiments are also disclosed.

In particular, the present device may be implemented in users in need of treatment of soft tissue in an extremity, e.g., soft tissue which has been subjected to strain, swelling, irritation or injury. For example, the present device may be used for therapy in an individual suffering from an upper extremity musculoskeletal disorder of the arm. In particular, device 10 may be used for treatment of carpal tunnel syndrome, tendonitis, epicondylitis (i.e., tennis elbow), and hand-arm vibration syndrome. Device 10 may be used to aid in therapeutic motions or stretches to rehabilitate injured soft tissue. Through use of the device, it is possible to stretch or strengthen muscles or tendons in the arm (e.g., muscles and tendons in communication with one or more of the elbow, forearm, wrist or fingers) without necessitating aid from the user's other arm. Thus, the presently disclosed devices provide an effective solution to allow for simultaneous rehabilitation of soft tissue while also freeing the use of the user's other hand for non-therapeutic purposes.

Reference is made in detail to the exemplary embodiments of the present disclosure, which are illustrated in the accompanying figures. Turning to the figures, there are illustrated components of a personal care applicator device 10.

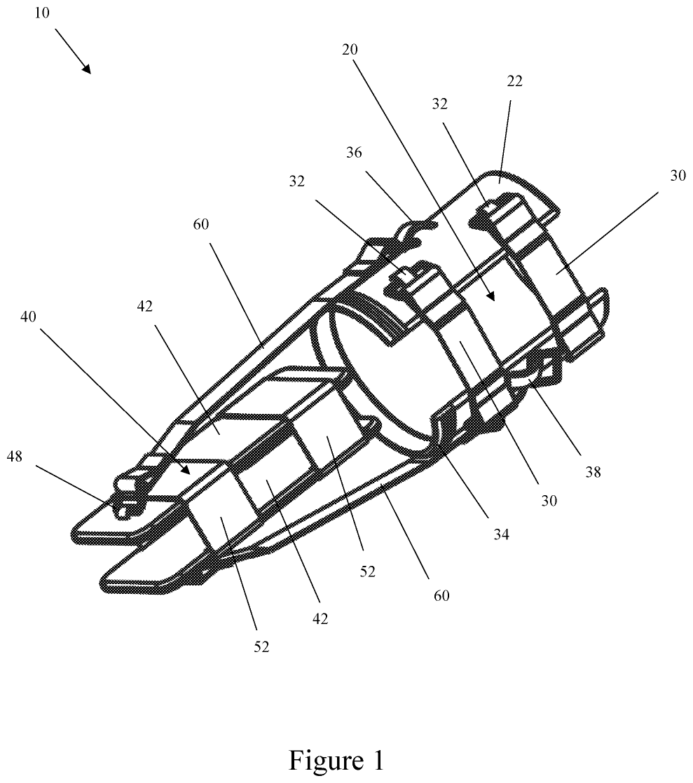

Accordingly, disclosed herein is a therapeutic device 10, which comprises a brace 20 and a digit immobilizer 40, which are in communication with one another through at least one strap extending from the brace 20 to the digit immobilizer 40, as shown in FIG. 1. Therapeutic device 10 optionally further comprises an upper arm support 80 positionable on an arm proximal to brace 20 (i.e., in such uses where brace 20 is placed over a user's forearm) and may be secured to a proximal end of brace 20. Therapeutic device 10 is placed upon an arm of a user suffering from soft tissue damage such as strain, swelling, irritation or injury.

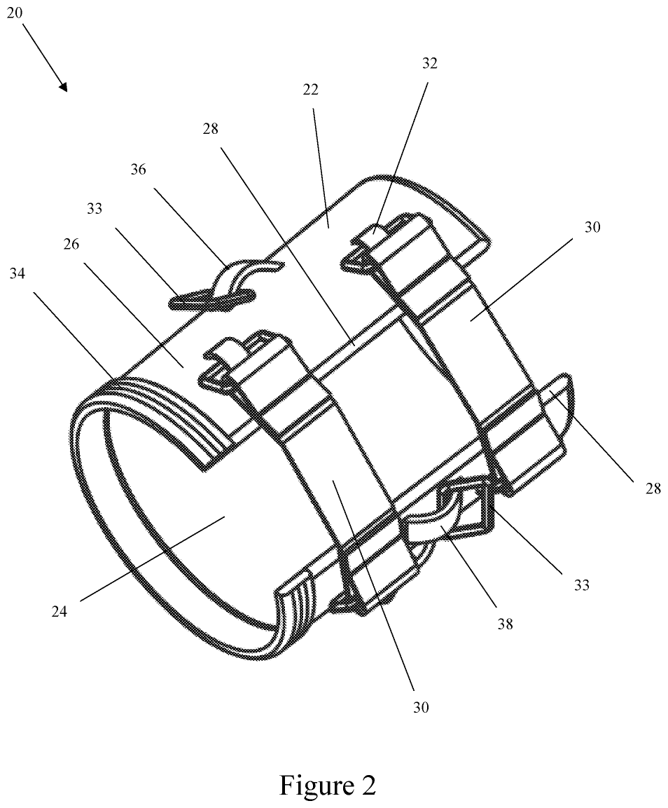

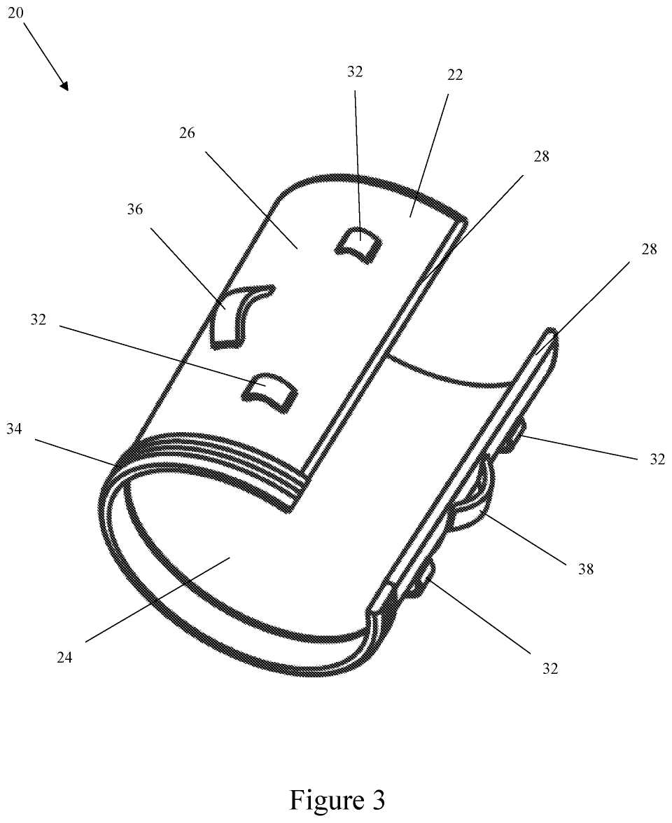

Brace 20 comprises a curved body 22 that partially or completely encompasses a portion of an arm. In various embodiments, brace 20 encompasses a user's arm proximal to the elbow, e.g., a portion of the arm surrounding the user's bicep. Alternatively, brace 20 is sized to encompass a user's forearm partially or completely. As shown for example, in either FIG. 2 or 3, curved body 22 of brace 20 comprises a C-shaped cross section. Curved body 22 comprises an inner surface 24 facing toward the skin, such that when worn, inner surface 24 abuts the skin of a user's arm. Outer surface 26 is positioned opposite inner surface 24 such that when worn, outer surface 26 faces away from the skin of a user's arm. As shown, curved body 22 arcs between oppositely positioned ends 28 which are not in contact with one another and extends for a length from a proximal end to a distal end. That is, curved body 22 comprises a gap at opposing ends 28 of the curved body 22. In various embodiments, curved body 22 comprises a uniform curvature between the two ends 28 and defines a gap therebetween. It is envisioned that in various embodiments, curved body 22 is monolithically formed to retain its structure and curvature such that curved body 22 is defined by a substantially consistent radius.

Brace 20 comprises at least one fastener 30 spanning the gap of curved body 22 to secure brace 20 firmly in place on a user's arm. In various embodiments, the at least one fastener 30 is adjustable to constrict or expand the gap. That is, in various embodiments, the at least one fastener 30 is adjustable to restrict or expand the radius of the curved body. It is envisioned that brace 20 may comprise more than a single fastener 30. In other embodiments, brace 20 comprises from two to five fasteners 30. In certain embodiment, fastener 30 may have a single attachment point at each end. However, in other embodiments, fastener 30 may have a single attachment point at a first end that branches into multiple attachment points at the opposite end. As shown in FIG. 2, brace 20 comprises two fasteners 30 in certain embodiments. Each fastener 30 is attached to brace 20 at an engagement loop 32, as shown best in FIG. 3. As shown for example in FIG. 2, fastener 30 may be in communication with engagement loop 32 via a hook 33, through which the fastener 30 may be passed.

Fasteners 30 may be made from a variety of materials, such as a durable fabric (e.g., nylon). However, fasteners 30 may be made from any material capable of exerting a tightening force on curved body 22. The at least one fastener 30 may be tightened by a variety of means. For example, fastener 30 may comprise a hook and loop material, adjustable snaps, or adhesive to allow its length to be adjusted easily while maintaining a tight hold. Hook 33 may comprise various strap hardware items used to adjust the length of fastener 30, such as slide clips, loops, rings, strap adjusters. In some embodiments, fastener 30 is affixed to one end 28 of curved body 22, and a cambuckle is affixed to the opposite end 28 to clasp down on fastener 30 to maintain fastener 30 at a suitable length. In some embodiments, fastener 30 may alternatively be made from a ridged plastic to ratchet into a catch such that fastener 30 may be tightened easily without loosening. In some embodiments, fastener 30 may utilize a twistable or ratcheting mechanism to adjust its length, such that when the mechanism is twisted, the length of fastener 30 is changed and secured in place. In some embodiments, the at least one fastener 30 comprises sufficient elastic character to enclose the opening for a tight fit around a user's arm. In such embodiments, fastener 30 may be permanently affixed to brace 20 but allow for the brace to be stretched to fit a user's arm.

In various embodiments, curved body 22 is composed of a pliable material (e.g., plastic). Thus, it is envisioned that brace 20 may be made from any material that enables it to retain its solid shape, while allowing curved body 22 some amount of flexibility to accommodate arms of various thicknesses. For example, curved body 22 may be made from a material selected from thermoplastic elastomer, polystyrene, polyurethane (e.g., thermoplastic polyurethane), neoprene, silicone, rubber, nylon, acrylonitrile butadiene styrene, acetates, acrylates, ceramics, fiberglass, epoxies, polyethylene, polyoxymethylene, polypropylene, polyvinylchloride, polycarbonate, or composites thereof. In other embodiments, however, curved body 22 may be made from a fabric material, e.g., a shape-fitting fabric, to allow for a greater degree of flexibility in fitting human arms of varying sizes.

In some embodiments, brace 20 comprises a cushion member 34 at one or both of the proximal and distal ends of the brace. In some embodiments, as shown in FIGS. 2 and 3, brace 20 comprises cushion member 34 at the distal end curved body 22 of brace 20. The cushion member 34 is comprised of a material that enhances the comfort of brace 20 for a person using therapeutic device 10. Cushion member 34 spans around the circumferential length of the curved body 22 of brace 20 and terminates at the ends 28 of brace 20. Thus, the gap defined by ends 28 of brace 20 extends equally between the endpoints of cushion member 34.

Cushion member 34 may be made from a shape-retaining material (e.g., plush, memory foam or silicone material) so as to conform to the contours of a human arm. In some embodiments, for example as best shown in FIG. 3, cushion member 34 is ribbed for increased flexibility. In further embodiments, cushion member 34 is coated in a grip enhancing material so as to aid in preventing brace 20 from sliding while device 10 is in use.

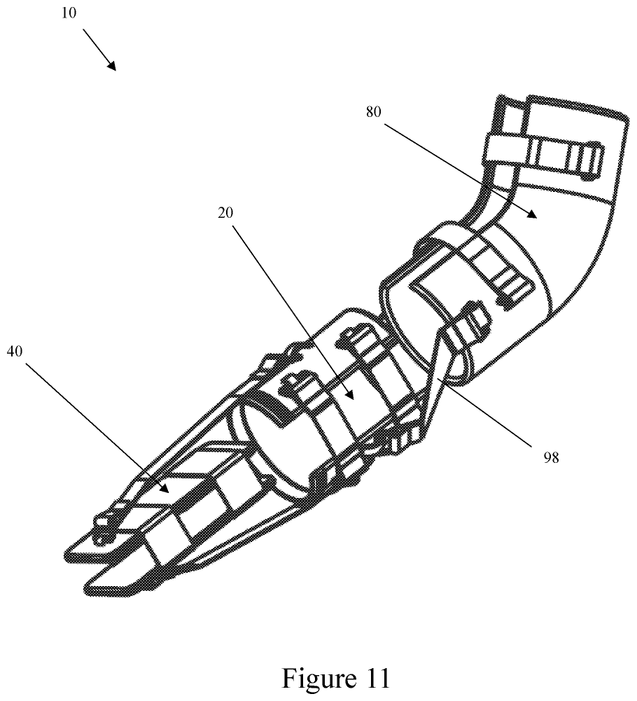

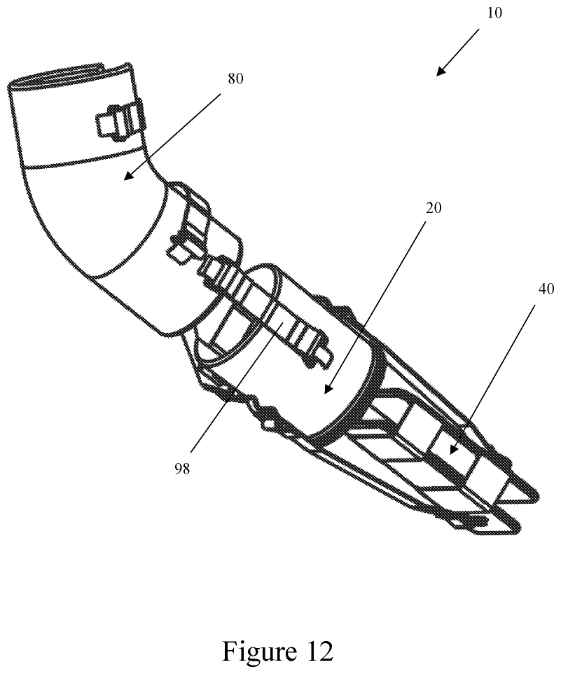

Outer surface 26 of curved body 22 further comprises a series of attachment points for maintaining communication with the other components of therapeutic device 10. That is, the attachment points are present to receive straps, fasteners or connection members in communication with the digit immobilizer 40 and the upper arm support 80. Outer surface 26 comprises at least one strap engagement loop 36 to receive a strap in communication with a plate 42 of digit immobilizer 40. In particular embodiments, for example as shown in FIG. 1, outer surface 26 comprises two oppositely positioned strap engagement loops 36 to receive a pair of straps, each in communication with a respective plate 42 of digit immobilizer 40. As shown, strap engagement loops 36 are positioned on outer surface 26 such that a first strap engagement loop 36 is medially-facing and a second strap engagement loop 36 is laterally-facing when placed onto an arm. In this way, the medially-facing strap engagement loop 36 is medially aligned with a corresponding engagement loop 48 of a plate 42, and the laterally-facing strap engagement loop 36 is laterally aligned with a corresponding engagement loop 48 of a plate 42. In certain embodiments, outer surface 26 further comprises at least one connecting member loop 38 to receive a connecting member 98 in communication with upper arm support 80. In particular embodiments, for example as shown in FIGS. 11 and 12, outer surface 26 comprises two oppositely positioned connecting member loops 38 to receive a pair of connecting member 98, each in communication with upper arm support 80. The at least one connecting member loop 38 on curved body 22 may be placed anywhere along outer surface 26, however, the connecting member loops 38 are shown in the figures as being orthogonally disposed on outer surface 26 relative to strap engagement loops 36 to prevent straps and connecting members 98 from interfering with one another. However, it is envisioned that in alternative embodiments, the straps are permanently affixed to outer surface 26 of brace 20.

The devices of the present disclosure further comprise a digit immobilizer 40, which comprises two plates 42 as well as at least one band 52. Digit immobilizer 40 encloses over opposing sides of a hand such that during use it is located distal to brace 20. When positioned on a hand, the digit immobilizer 40 exerts stabilizing pressure on a user's hand to prevent articulation of the digits and associated joints. That is, when a hand is enclosed between plates 42, motion of the distal interphalangeal joints, proximal interphalangeal joints, metacarpophalangeal joints is substantially restricted or entirely prevented. This restriction of motion is beneficial to maintain appropriate posture of the hand for stretching positions and strengthening motions which can be carried out through use of therapeutic device 10.

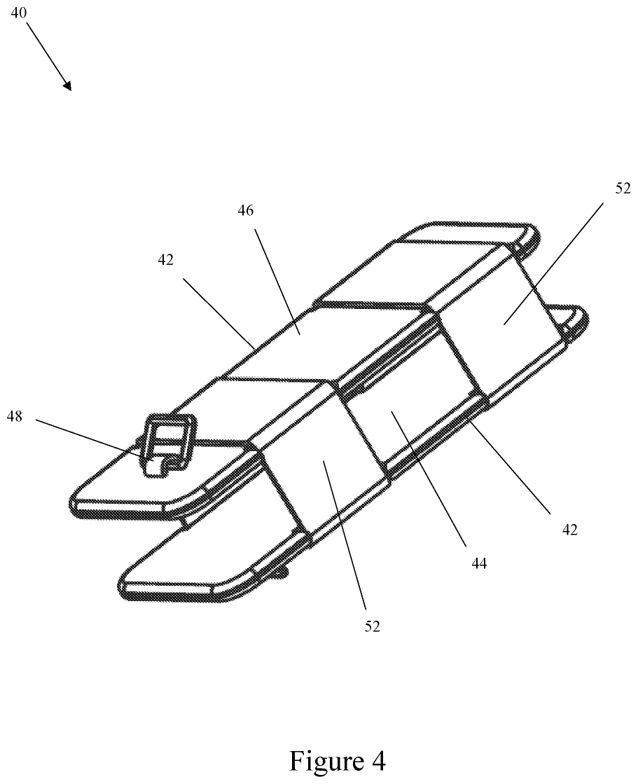

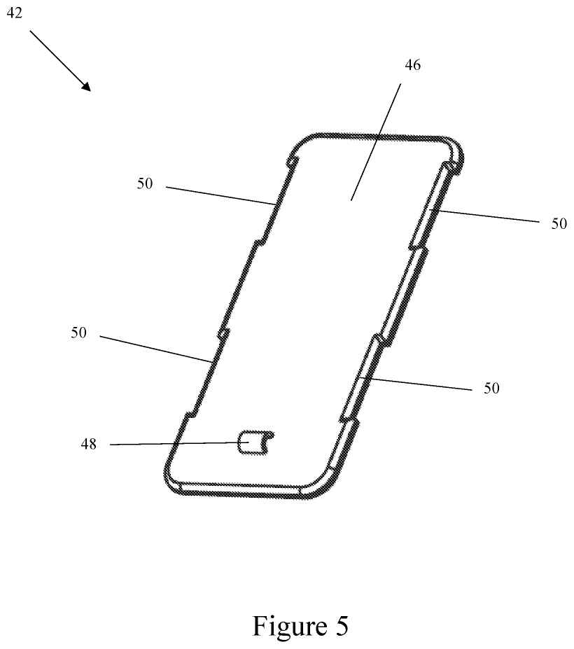

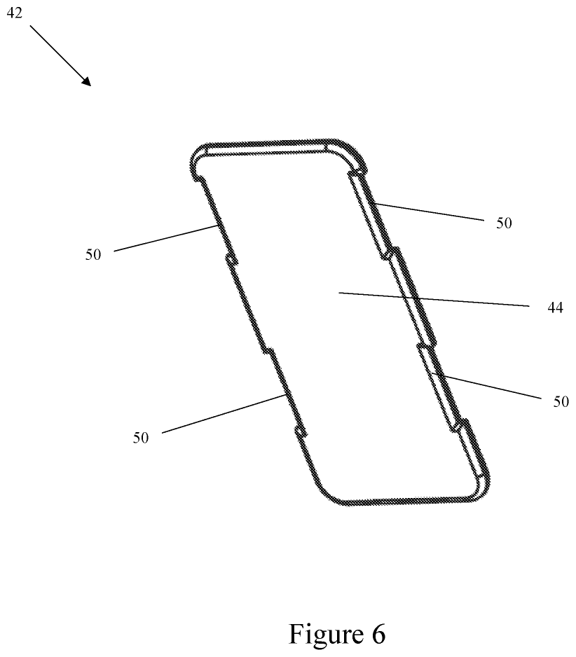

Digit immobilizer 40 comprises two plates, each having a substantially identical structure. In certain embodiments, for example as show in FIGS. 4, 5 and 6, plates 42 of digit immobilizer 40 are each substantially planar and comprise an inner surface 44 (i.e., surface contactable against the skin) and an outer surface 46 (i.e., surface opposite the inner surface). In various embodiments, plates 42 of digit immobilizer 40 are each defined by a square, rectangular, rounded, or irregularly shaped perimeter. As shown best in FIGS. 5 and 6, plates 42 of digit immobilizer 40 are each defined by a substantially rectangular perimeter. Each plate 42 has a size which generally conforms to the dimensions of a human hand. While plates 42 as shown in the Figures comprise identical structures, it is envisioned that plates 42 may comprise varied sizes, shapes and orientations.

In various embodiments, plates 42 are made from a non-flexible, rigid material, e.g., wood, metal, ceramic, glass or plastics. In some embodiments, plate 42 is formed from or coated with a material which possesses a rubberized texture to keep the user's hand from slipping. For example, plates 42 may be made from or coated with silicone, rubber, paraffin wax, polyethylene or other such materials. Plates 42 may be comprised of a single unitary piece, which may be made by injection molding or other known methods in the art.

Both the inner surface 44 and outer surface 46 of each plate 42 is substantially planar. Inner surface 44 is substantially flat and free of any protuberances or protrusions, while outer surface 46 comprises an engagement loop 48 to receive a strap in communication with brace 20. Digit immobilizer 40 comprises a medial plate 42 disposable with the palm of a hand and a lateral plate 42 disposable with the back of the hand. The inner surface 44 of both medial plate 42 and lateral plate 42 face inward so as to contact the skin during use, while the outer surface 46 of both medial plate 42 and lateral plate 42 face outward and away from the skin such that engagement loop 48 on each respective outer surface 46 is accessible for connection with brace 20. Medial plate 42 and lateral plate 42 as shown are substantially identical, and for the purposes of this specification may be interchangeably interpreted based solely on the orientation of the plates when outfitted around a hand.

Plates 42 of the digit immobilizer 40 comprise a plurality of grooves 50 along the perimeter of the plates 42 to receive at least one band 52 (e.g., one band 52 or two bands 52). Grooves 50 are configured to accommodate bands 52 that apply pressure between the plates 42 against the hand so as to keep plates 42 disposed firmly in place during use. As shown plate 42 comprises a plurality of grooves 50 disposed along its perimeter. The number of grooves 50 corresponds with the number of bands 52 in use. Grooves 50 are present in pairs on opposite sides along the perimeter of each plate 42. Thus, as shown in FIGS. 4-6, each plate comprises four grooves 50 to accommodate two bands 52.

It is envisioned that bands 52 may be adjustable or elastic. In embodiments in which bands 52 are adjustable, they are comprised of a durable fabric material (e.g., nylon), and may have their length adjusted through various means. For example, a band 52 may comprise a hook and loop material, adjustable snaps, or adhesive to allow its length to be adjusted easily while maintaining plates 42 in a tight fit around a hand. Various items may be used to adjust the length of an adjustable band 52, such as slide clips, loops, rings, strap adjusters. In other embodiments, bands 52 are comprised of an elastic material and have sufficient elastic resistance to prevent articulation of the digits during use when received within the grooves of the medial and lateral plates. Bands 52 engage with grooves 50 to prevent sliding along the perimeter of plates 42, while also placing inward pressure between the inner surfaces of plates and the skin of the hand (i.e., the palm and back of the hand, respectively).

Outer surface 46 of each plate 42 comprises at least one engagement loop 48 to receive a strap in communication with brace 20. For example, as shown in FIG. 1, each outer surface 46 of oppositely positioned plates 42 comprises an engagement loop 48 to receive a pair of straps, each in communication with opposite sides of brace 20. As shown, engagement loops 48 are positioned on outer surface 46 such that a first engagement loop 48 on a medial plate 42 is medially-facing and a second engagement loop 48 on a lateral plate 42 is laterally-facing when placed onto a hand. In this way, the medially-facing engagement loop 48 is medially aligned with a corresponding strap engagement loop 36 of brace 20, and the laterally-facing engagement loop 48 is laterally aligned with a corresponding strap engagement loop 36 of brace 20.

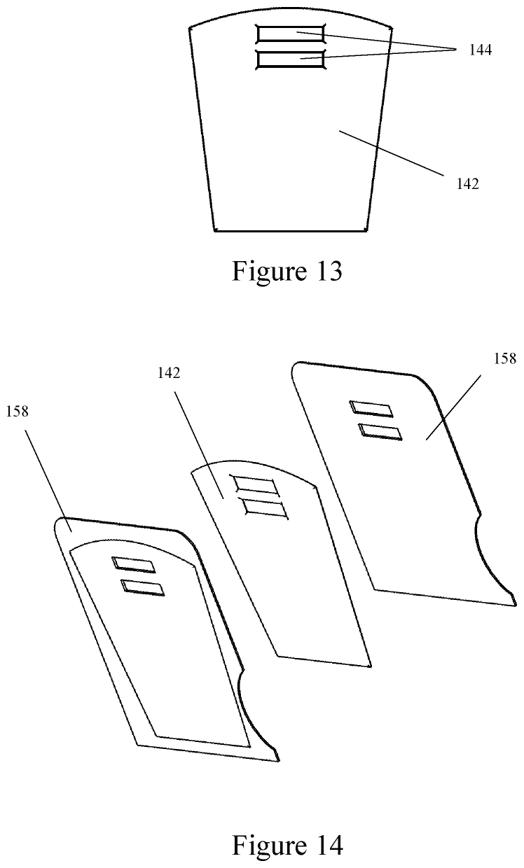

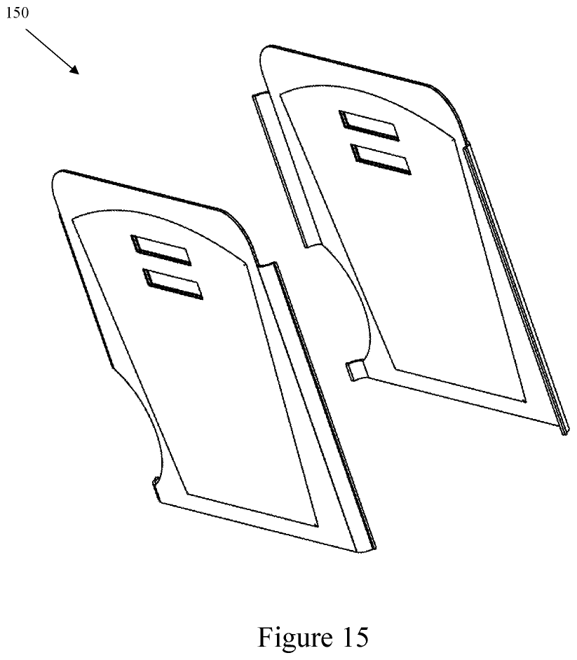

In a further embodiment, as shown in FIGS. 13 and 14, the digit immobilizer 150 comprises a pair of plates 142. each having a substantially identical structure. Plates 142 are each substantially planar and comprise an inner surface and an outer surface, and are each defined by a square, rectangular, rounded, or irregularly shaped perimeter. Each plate 142 has a size which generally conforms to the dimensions of a human hand. While plates 142 as shown in the Figures comprise identical structures, it is envisioned that plates 42 may comprise varied sizes, shapes and orientations. In various embodiments, plates 142 are made from a non-flexible, rigid material, e.g., wood, metal, ceramic, glass or plastics. Plates 42 may be comprised of a single unitary piece, which may be made by injection molding or other known methods in the art.

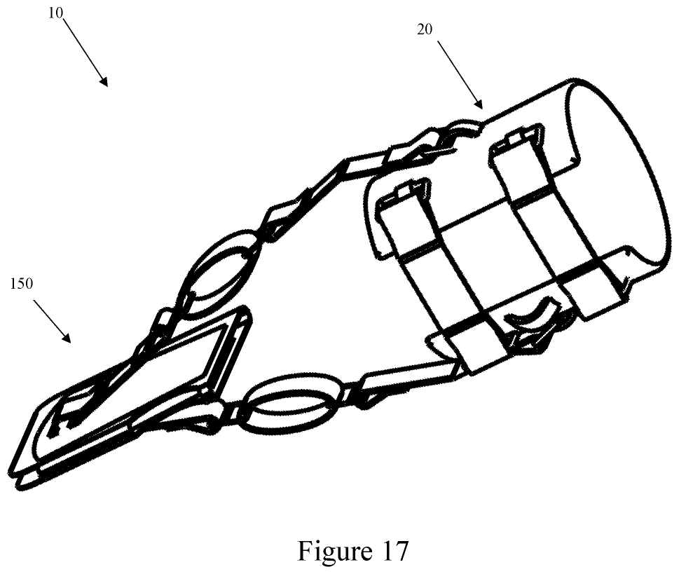

Both the inner surface and outer surface of each plate 142 is substantially planar. However, plate 142 comprises a pair of slots 144 to receive a strap in communication with brace 20, for example as shown in FIG. 17. Digit immobilizer 150 comprises a medial plate 142 positioned adjacent to the palm of a hand and a lateral plate 142 positioned adjacent with the back of the hand. Medial plate 142 and lateral plate 142 as shown are substantially identical, and for the purposes of this specification may be interchangeably interpreted based solely on the orientation of the plates when outfitted around a hand.

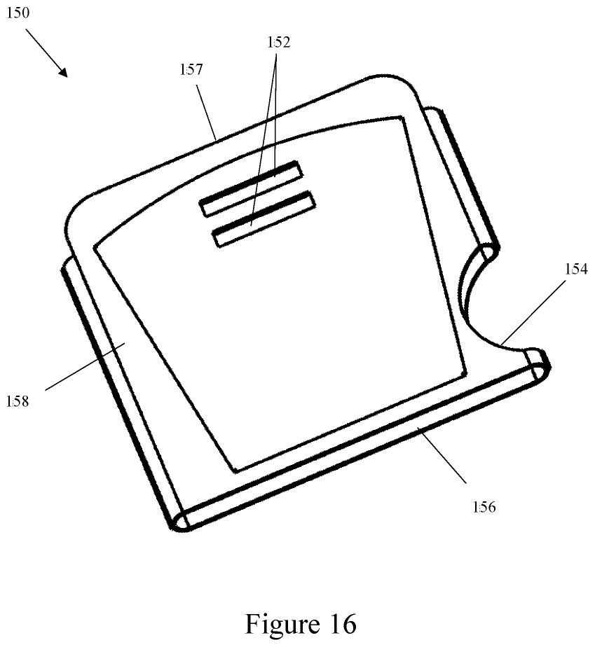

As shown in FIGS. 14-16, plates 142 may be received within a glove 150 to fit directly to a user's hand. Glove 150 is comprised of material 158, which may be a fabric, leather or a leatherette. As best shown in FIG. 14, glove 150 may comprise a multitude of layers (e.g., two layers) each comprised of material 158 and between which plate 142 is received. The two layers of material 158 may be attached to one another through various means, including sewn or heatpressed. In some embodiments, the main body of glove 150 is comprised of a single layer of material 158, but a second layer of material 158 is used to create a pocket to receive and secure plate 142. In other embodiments, plates 142 are removeable from the pockets formed between layers of material 158, e.g., through a slot at any end of the pocket. As best shown in FIG. 15, glove 150 medially receives receive a first plate 142 to position against the palm, and laterally receives a second plate to position against the back of the hand.

Glove 150 comprises a structure that generally conforms to that of a human hand. Glove 150 comprises a pair of slots 152 on both the medial side and the lateral side of the glove, where said slots 152 are positioned to overlap with slots 144 of the plates 142 received within. Slots 152 and 144 allow for a strap to directly connect with plate 142 through the material of glove 150. In order to accept a human hand, glove 150 comprises a proximal opening 156 and a distal opening 157 positioned on opposite ends of glove 150. Glove 150 further includes a thumb hole 154 positioned to allow passage of a user's thumb. During use, glove 150 is fit over a hand such that the thumb passes through thumb hole 154, the remaining fingers pass through proximal opening 157, and proximal opening 156 is positioned adjacent to the wrist. Further discussion herein involving plate 42 shall be understood to encompass embodiments including plate 142 in equivalent fashion.

Therapeutic device 10 further comprises at least one strap which extends from a distal end of curved body 22 of brace 20 to one of the plates 42 of digit immobilizer 40. It is envisioned that device 10 may comprise one or two straps extending between curved body 22 and digit immobilizer 40. In some embodiments, for example, as shown in FIG. 1, device 10 comprises a pair of straps extending from a distal end of curved body 22 of brace 20 to one of the plates 42 of digit immobilizer 40. In such embodiments comprising two straps, the first strap extends from a distal end of the curved body 22 on a lateral-facing portion of brace 20 to a lateral plate 42, and the second strap extends from a distal end of the curved body 22 on a medial-facing portion of brace 20 to a medial plate 42. The type of strap used may be changed depending on the desired purpose during use. That is, the at least one strap is either a static soft tissue-stretching strap 60 or a strengthening strap 64.

As illustrated in FIGS. 1, 11 and 12, the at least one strap may be a soft tissue-stretching strap 60 which has an adjustable length. The soft tissue-stretching strap 60 is used when the desired therapeutic effect of device 10 is stretching soft tissue in the arm, e.g., medial or lateral muscle groups in the forearm or associated tendons. In many embodiments, static soft tissue-stretching strap 60 is substantially or completely non-elastic. Static soft tissue-stretching strap 60 has a fixable length such that when in place extending between brace 20 and the digit immobilizer 40, one of extension or flexion of the wrist is limited.

It is envisioned that the overall length of soft tissue-stretching strap 60 is adjustable to be lengthened or shortened. When shortened, soft tissue-stretching strap 60 restricts the wrist from articulation toward flexion or extension. Conversely, when lengthened, soft tissue-stretching strap 60 allows free articulation of the wrist toward flexion or extension. If soft tissue-stretching strap 60 is adjusted to be shortened on the medial side of the arm (e.g., shortened while attached to the outer surface of medial plate 42 and a medially-facing position on curved body 22), the wrist is moved and maintained in a flexion position. In certain embodiments, a second soft tissue-stretching strap 60 is present attached oppositely on the lateral side of the arm and is made longer to accommodate wrist in the flexion position. If soft tissue-stretching strap 60 is adjusted to be shortened on the lateral side of the arm (e.g., shortened while attached to the outer surface of lateral plate 42 and a laterally-facing position on curved body 22), the wrist is moved and maintained in an extension position. In certain embodiments, a second soft tissue-stretching strap 60 is present attached oppositely on the medial side of the arm and is made longer to accommodate wrist in the extension position.

The at least one soft tissue-stretching strap 60 may be tightened by variety of means. For example, soft tissue-stretching strap 60 may comprise a hook and loop material, adjustable snaps, or adhesive to allow its length to be adjusted easily while maintaining a tight hold. In some embodiments, strap 60 may utilize a twistable or ratcheting mechanism to adjust the length, such that when the mechanism is twisted, the length of strap 60 is changed. Soft tissue-stretching strap 60 may be affixed to one or both of brace 20 and digit immobilizer 40 through various strap hardware items, such as slide clips, loops, rings, strap adjusters. For example, a first end of soft tissue-stretching strap 60 may be affixed to one of brace 20 or digit immobilizer 40 and a second end may be slipped through a loop on the other of brace 20 or digit immobilizer 40. The second end may be adjusted to a desired length and pinned to itself to firmly hold the desired length between brace 20 and digit immobilizer 40. In some embodiments, soft tissue-stretching strap 60 is affixed to one of brace 20 or digit immobilizer 40, and a cambuckle is affixed to the other to clasp down on soft tissue-stretching strap 60 to maintain soft tissue-stretching strap 60 at a suitable length. In some embodiments, soft tissue-stretching strap 60 may alternatively be made from a ridged plastic to ratchet into a catch such that soft tissue-stretching strap 60 may be tightened easily without loosening.

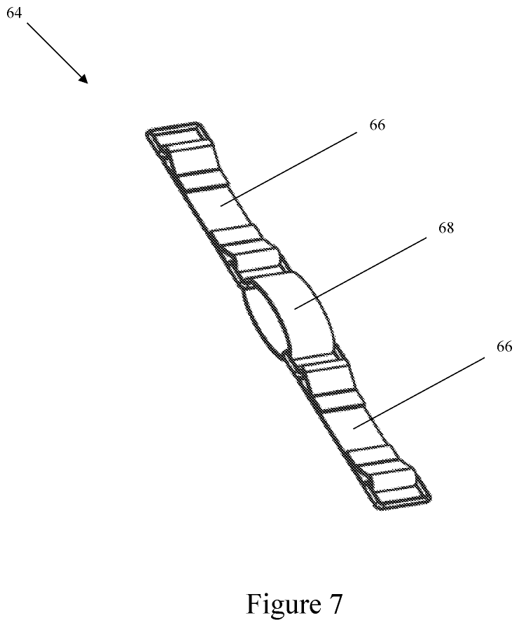

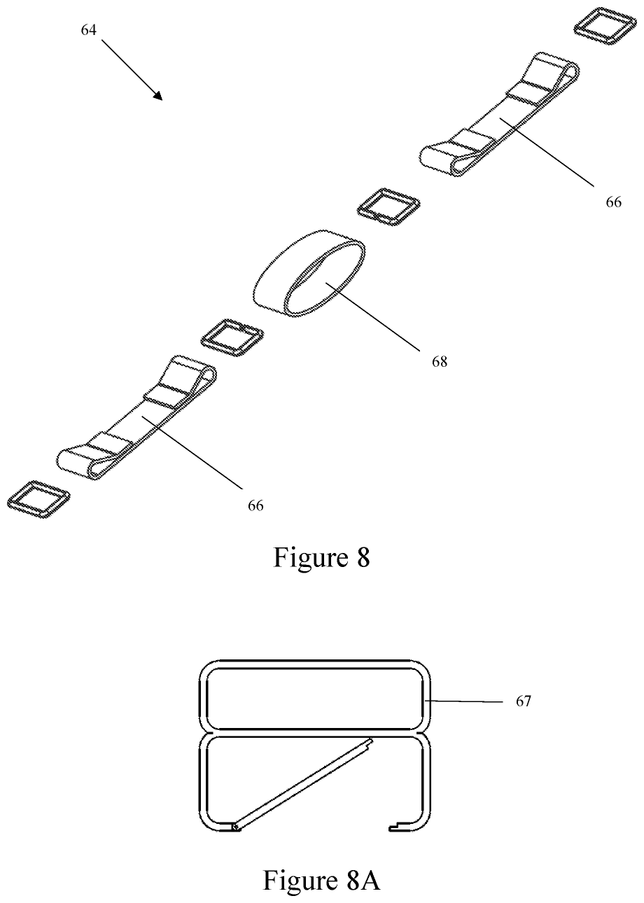

If the desired use of the therapeutic device 10 is to strengthen soft tissue in the arm, e.g., strengthen the medial or lateral muscle groups in the forearm or associated tendons, then a strengthening strap 64 may be used in place of soft tissue-stretching strap 60. As shown for example in either of FIG. 7 or 8, strengthening strap 64 comprises an elastic resistance member 68, which may be made from a durable elastic material (e.g., rubber). The elastic resistance member 68 may be modified or replaced to impart varying degrees of elasticity upon strengthening strap 64. It is possible to achieve a variance in the degree of elasticity through, e.g., modification of the material of elastic resistance member 68 or changing thickness of the material. In certain embodiments, strengthening strap 64 comprises an elastic resistance member 68 having an elastic resistance such that when strengthening strap 64 is in place extending between brace 20 and digit immobilizer 40, resistance force is placed on the wrist against one of extension or flexion.

As shown in FIG. 7, strengthening strap 64 comprises elastic resistance member 68, which is engaged with and running between opposite tethers 66. In various embodiments, tethers 66 are substantially or completely non-elastic, and all elasticity of strengthening strap 64 derives from elastic resistance member 68. As best shown in the exploded view of FIG. 8, tethers 66 are enclosed at both ends by rings, e.g., rectangular rings, to allow for hinging movement during strengthening exercises. In some embodiments, tethers 66 are permanently affixed to the rings. However, the rings joining tethers 66 to the elastic resistance member 68 may be slotted or otherwise manipulable to allow the elastic resistance member 68 to be swapped for another elastic resistance member 68 having a different degree of elasticity. For example, as shown in FIG. 8A, the tether may have a structure as shown by tether 67, which comprises a hinging surface to allow for easy removal and replacement of elastic resistance member 68 or even for attachment of a soft tissue-stretching strap 60. In this way, a user may easily switch between strengthening and stretching modes of use while keeping the device in place on the arm

During use, strengthening strap 64 may be stretched through motion of the wrist opposing the resistance force, i.e., such that the lateral muscles responsible for extension or the medial muscles responsible for flexion are strengthened through resistance training. That is, when strengthening strap 64 is in place extending between brace 20 and the digit immobilizer 40, resistance force is placed on the wrist against one of extension or flexion. If strengthening strap 64 is placed on the medial side of the arm (e.g., attached to the outer surface of medial plate 42 and a medially-facing position on curved body 22), then the wrist will be biased through elastic resistance toward a flexion position. Conversely, if strengthening strap 64 is placed on the lateral side of the arm (e.g., attached to the outer surface of lateral plate 42 and a laterally-facing position on curved body 22), then the wrist will be biased through elastic resistance toward an extension position. The elasticity of elastic resistance member 68 of strengthening strap 64 allows for the user to move the wrist toward the flexion or extension position, as the case my be, with sufficient force as to overcome the elastic bias. Through repetitive motions, the muscles in the forearm are strengthened. It is possible that a soft tissue-stretching strap 60 may be affixed opposite strengthening strap 64. In such configurations, soft tissue-stretching strap 60 is in a lengthened adjustment to allow for a desired degree of motion during the strengthening exercise.

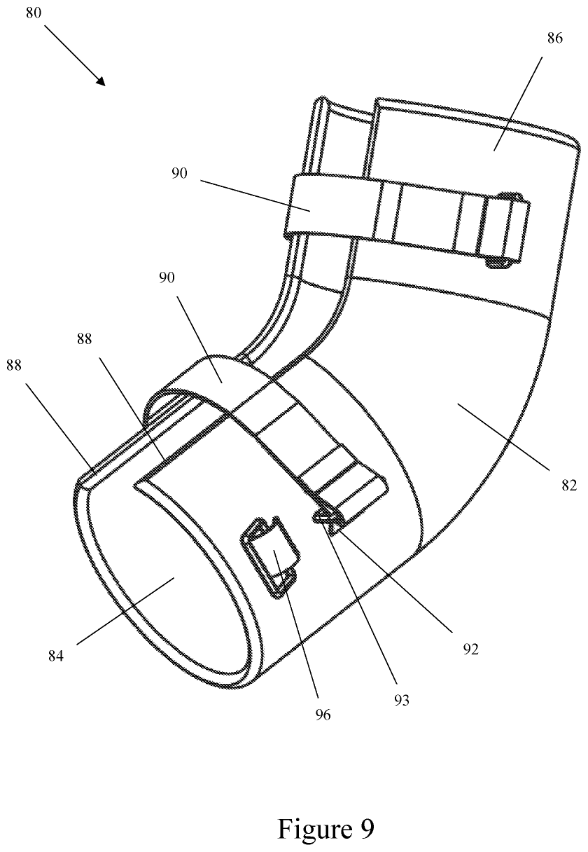

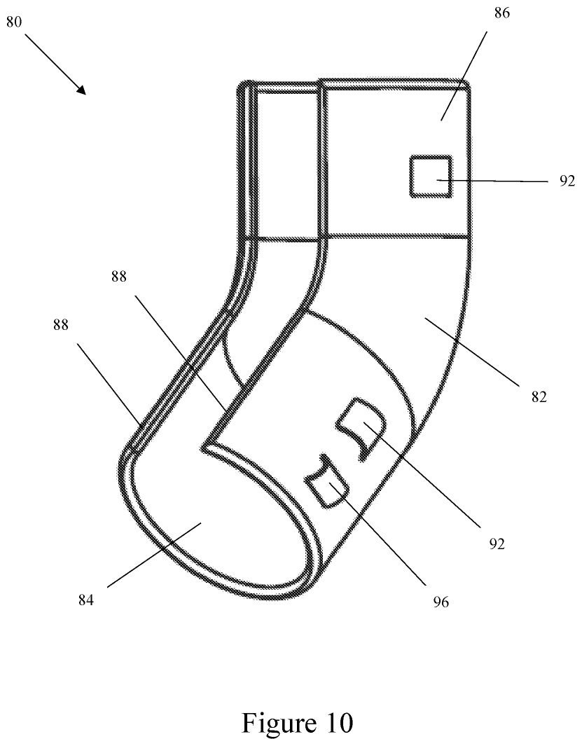

In certain embodiments, therapeutic device 10 further comprises an upper arm support 80. As shown in FIGS. 9 and 10, upper arm support 80 may be angled to accommodate a neutral positioning of the elbow. However, in other embodiments, upper arm support may comprise a shape to accommodate only a portion of the arm proximal to the elbow.

Upper arm support 80 comprises a curved body 82 that partially or completely encompasses a portion of an arm. In various embodiments, upper arm support 80 is sized to encompass a portion of a user's upper forearm and elbow partially or completely. As shown for example, in either FIG. 9 or 10, curved body 82 of upper arm support 80 comprises a C-shaped cross section. Curved body 82 comprises an inner surface 84 facing toward the skin, such that when worn, inner surface 84 abuts the skin of a user's arm. Outer surface 86 is positioned opposite inner surface 86 such that when worn, outer surface 86 faces away from the skin of a user's arm. As shown, curved body 82 arcs between oppositely positioned ends 88 which are not in contact with one another, and extends for a length from a proximal end to a distal end. That is, curved body 82 comprises a gap at opposing ends 88 of the curved body 82. In various embodiments, curved body 82 comprises a uniform curvature between the two ends 88 and defines a gap therebetween. It is envisioned that in various embodiments, curved body 82 is monolithically formed to retain its structure and curvature such that curved body 82 is defined by a substantially consistent radius.

Upper arm support 80 comprises at least one fastener 90 spanning the gap of curved body 82 to secure upper arm support 80 firmly in place on a user's arm. In various embodiments, the at least one fastener 90 is adjustable to constrict or expand the gap. That is, in various embodiments, the at least one fastener 90 is adjustable to restrict or expand the radius of the curved body. It is envisioned that upper arm support 80 may comprise more than a single fastener 90. In other embodiments, upper arm support 80 comprises from two to five fasteners 90. In certain embodiment, fastener 90 may have a single attachment point at each end. However, in other embodiments, fastener 90 may have a single attachment point at a first end that branches into multiple attachment points at the opposite end. As shown in FIG. 2, upper arm support 80 comprises two fasteners 90 in certain embodiments. Each fastener 90 is attached to upper arm support 80 at an engagement loop 92, as shown best in FIG. 10. As shown for example in FIG. 9, fastener 90 may be in communication with engagement loop 92 via a hook 93, through which the fastener 90 may be passed.

Fasteners 90 may be made from a variety of materials, such as a durable fabric (e.g., nylon). However, fasteners 90 may be made from any material capable of exerting a tightening force on curved body 82. The at least one fastener 90 may be tightened by variety of means. For example, fastener 90 may comprise a hook and loop material, adjustable snaps, or adhesive to allow its length to be adjusted easily while maintaining a tight hold. Hook 93 may comprise various strap hardware items used to adjust the length of fastener 90, such as slide clips, loops, rings, strap adjusters. In some embodiments, fastener 90 is affixed to one end 88 of curved body 82, and a cambuckle is affixed to the opposite end 88 to clasp down on fastener 90 to maintain fastener 90 at a suitable length. In some embodiments, fastener 90 may alternatively be made from a ridged plastic to ratchet into a catch such that fastener 90 may be tightened easily without loosening. In some embodiments, fastener 90 may utilize a twistable or ratcheting mechanism to adjust its length, such that when the mechanism is twisted, the length of fastener 90 is changed and secured in place. In some embodiments, the at least one fastener 90 comprises sufficient elastic character to enclose the opening for a tight fit around a user's arm. In such embodiments, fastener 90 may be permanently affixed to upper arm support 80 but allow for the brace to be stretched to fit a user's arm.

In various embodiments, curved body 82 is composed of a pliable material (e.g., plastic). Thus, it is envisioned that upper arm support 80 may be made from any material that enables it to retain its solid shape, while allowing curved body 82 some amount of flexibility to accommodate arms of various thicknesses. For example, curved body 82 may be made from a material selected from thermoplastic elastomer, polystyrene, polyurethane (e.g., thermoplastic polyurethane), neoprene, silicone, rubber, nylon, acrylonitrile butadiene styrene, acetates, acrylates, ceramics, fiberglass, epoxies, polyethylene, polyoxymethylene, polypropylene, polyvinylchloride, polycarbonate, or composites thereof. In other embodiments, however, curved body 82 may be made from a fabric material, e.g., a shape-fitting fabric, to allow for a greater degree of flexibility in fitting human arms of varying sizes.

Outer surface 86 of curved body 82 further comprises a series of attachment points for maintaining communication with the other components of therapeutic device 10. That is, the attachment points are present to receive connection members 98 in communication with brace 20. In certain embodiments, outer surface 86 further comprises at least one connecting member loop 96 to receive a connecting member 98 in communication with brace 20. For example, as shown in FIGS. 11 and 12, outer surface 86 comprises two oppositely positioned connecting member loops 96 to receive a pair of connecting members 98, each in communication with brace 20. The at least one connecting member loop 96 on curved body 82 may be placed anywhere along outer surface 86, however, the connecting member loops 96 must be aligned with the connecting member loops 38 of brace 20.

Connection members 98 may be adjustable or elastic. In embodiments in which connection members 98 are adjustable, they are comprised of a durable fabric material (e.g., nylon), and may have their length adjusted through various means in order to adjust the length of connection between brace 20 and upper arm support 80. For example, a connection member 98 may comprise a hook and loop material, adjustable snaps, or adhesive to allow its length to be adjusted easily. Various items may be used to adjust the length of an adjustable connection member 98, such as slide clips, loops, rings, strap adjusters. In other embodiments, connection member 98 are comprised of an elastic material and have sufficient elastic resistance to maintain orientation and distance between brace 20 and upper arm support 80.

In various embodiments, the presently disclosed devices are useful in the treatment of soft tissue in the arm. In some embodiments, the present device is useful in the treatment of tendons, ligaments, or muscles in the arm. In some embodiments, the device is useful in the treatment of an upper extremity musculoskeletal disorders of the arm. In some embodiments, the device is useful in the treatment of carpal tunnel syndrome, tendonitis, epicondylitis (i.e., tennis elbow), and hand-arm vibration syndrome.

During use, the therapeutic devices 10 as disclosed herein may be used in a therapeutic method for rehabilitating soft tissue of the human body. In general, this method includes a first step of providing a therapeutic device 10 (e.g., a device comprising a brace 20 comprising a curved body 22 that partially or completely encompasses a portion of an arm; and a digit immobilizer 40 comprising two plates 42 to receive a hand in a flat position, wherein the brace 20 and the digit immobilizer 40 are in communication with one another through at least one strap extending from the body of the brace 20 to one of the plates 42 of the digit immobilizer 40). Once provided, the user proceeds to place the digit immobilizer 40 on a hand and place the brace 20 on a portion of the arm, in no particular order. The method also includes the step of attaching at least one strap between brace 20 and digit immobilizer 40 for a desired therapeutic effect. This desired effect may be either stretching or strengthening of soft tissue, e.g., soft tissue in the arm.

In order to place digit immobilizer 40 on a hand, bands 52 may be looped around plates 42 of digit immobilizer 40 and fasteners on brace 20 are tightened such that digit immobilizer 40 is firmly engaged with the user's hand and brace 20 is firmly engaged with the user's arm at either the forearm or the bicep. As discussed above, there are various possible configurations of straps which extend between brace 20 and digit immobilizer 40. In some embodiments, a first strap is attached at a first end to outer surface 46 of medial plate 42 and at a second end to a medial engagement loop 36 on brace 20. In addition or in alternative to the first strap, a second strap may be attached at a first end to outer surface 46 of lateral plate 42 and at a second end to a lateral engagement loop 36 on brace 20. It is thus envisioned that there may be a single strap extending between brace 20 and digit immobilizer 40 on a medial or lateral side of device 10, or there may be two oppositely disposed straps extending between brace 20 and digit immobilizer 40 on both medial and lateral sides of device 10.

The methods as described may further include the step of placing the upper arm support on a portion of the arm proximal to the brace, and further tightening fasteners on the upper arm support such that the upper arm support is firmly engaged with the user's upper forearm and elbow.

As discussed, device 10 is capable of applying a variety of therapeutic effects to the arm. Thus, in some embodiments, the therapeutic effect is stretching of soft tissue, and the strap is a soft tissue-stretching strap 60 as disclosed and described herein. Therapeutic device 10 may be used toward the desired therapeutic effect of stretching of the lateral muscle group of a user's forearm. In such embodiments, soft tissue-stretching strap 60 is attached at a first end to an outer surface 46 of a medial plate 42 and at a second end to a medial engagement loop 36 on the brace 20. The medially placed soft tissue-stretching strap 60 is then adjusted (i.e., shortened) to a certain length to lock the wrist in a forearm extensor stretching position. If a second strap is present in the lateral configuration, then that strap is extended to provide sufficient length to allow the wrist to be in the forearm extensor stretching position. Conversely, therapeutic device 10 may also be used toward the desired therapeutic effect of stretching of the medial muscle group of a user's forearm. In such embodiments, soft tissue-stretching strap 60 is attached at a first end to an outer surface 46 of a lateral plate 42 and at a second end to a lateral engagement loop 36 on the brace 20. The laterally placed soft tissue-stretching strap 60 is then adjusted (i.e., shortened) to a certain length to lock the wrist in a forearm flexor stretching position. If a second strap is present in the medial configuration, then that strap is extended to provide sufficient length to allow the wrist to be in the forearm flexor stretching position.

In other embodiments, the desired therapeutic effect is strengthening of muscle, and the strap is a strengthening strap 64 as disclosed and described herein. During use, strengthening strap 64 is stretched through motion of the wrist opposing the resistance force (i.e., such that the lateral forearm muscles (i.e., those responsible for extension) or the medial forearm muscles (i.e., those responsible for flexion) are strengthened through resistance training) applied from the elastic resistance of strengthening strap 64.

In some embodiments, the desired effect is strengthening of one or more of the medial forearm muscles, and the strap is attached to at a first end to outer surface 46 of lateral plate 42 and at a second end to a lateral engagement loop 36 on brace 20. Strengthening strap 64 comprises an elastic resistance member 68 with sufficient elastic resistance such that when strengthening strap 64 is in place extending between brace 20 and digit immobilizer 40, resistance force is placed on the wrist against flexion (i.e., toward the extension position). Once in place, a user may stretch strengthening strap 64 through motion of the wrist opposing the resistance force, in this case, through flexion of the wrist. This motion may be repeated as necessary to achieve a desired strengthening effect.

In some embodiments, the desired effect is strengthening of one or more of the lateral forearm muscles, and the strap is attached to at a first end to outer surface 46 of medial plate 42 and at a second end to a medial engagement loop 36 on brace 20. Strengthening strap 64 comprises an elastic resistance member 68 with sufficient elastic resistance such that when strengthening strap 64 is in place extending between brace 20 and digit immobilizer 40, resistance force is placed on the wrist against extension (i.e., toward the flexion position). Once in place, a user may stretch strengthening strap 64 through motion of the wrist opposing the resistance force, in this case, through extension of the wrist. This motion may be repeated as necessary to achieve a desired strengthening effect.

Further provided herein are kits for therapeutic device 10 for the manipulative treatment of soft tissue of the human body. In various embodiments, the kit comprises brace 20 having curved body 22 that partially or completely encompasses a portion of an arm (e.g., a portion of the forearm or upper arm around the bicep); digit immobilizer 40 comprising two plates 42 to receive a hand in a flat position; and a strap attachable at one end to curved body 22 of brace 20 and at the other end to one of plates 42 of digit immobilizer 40.

In various embodiments, the kit further comprises an upper arm support 80 having a curved body 82. The upper arm support 80 may have an angled portion to accommodate a natural position of the elbow. However, in other embodiments, upper arm support 80 is generally cylindrically shaped and configured to fit over a portion of the upper arm (i.e., a portion of the arm proximal to the elbow). In some embodiments, the kit may comprise multiple forms of the upper arm support 80, including an angled embodiment shown for example in FIG. 9 as well as a cylindrically shaped version of upper arm brace 80 configured to fit over a portion of the arm proximal to the elbow.

The kit may include fasteners for tightening one or both of brace 20 and upper arm support 80, e.g., fasteners 30 which are attachable to brace 20 and/or fasteners 90 which are attachable to upper arm support 80. In various embodiments, the fasteners may be removable from brace 20 and upper arm support 80 and thus may be packaged separately for the kit.

In various embodiments, the kit may comprise a variety of straps to enable multiple uses of therapeutic device 10, for example uses as a device for stretching soft tissue or strengthening muscle. Thus, in some embodiments, the kit further comprises a plurality of straps which are attachable at one end of curved body 22 of brace 20 and at the other end to one of plates 42 of digit immobilizer 40. As stated herein, the straps used with device 10 may take on several forms, including soft tissue-stretching straps 60 and strengthening straps 64. In some embodiments, the kit comprises a single soft tissue-stretching strap 60 and a single strengthening strap 64. However, it is envisioned that the kit may also comprise multiple of either the soft tissue-stretching strap 60 or strengthening strap 64. In some embodiments, the kit comprises a plurality of each of soft tissue-stretching straps 60 and strengthening straps 64.

The kit may comprise any number of soft tissue-stretching straps 60 and strengthening straps 64, some of which may be used as replacement straps. In various embodiments, the kit comprises a number of soft tissue-stretching straps 60 and strengthening straps 64 in an amount individually selected from one, two, three, four, five, six, seven, eight, nine or ten straps. In certain embodiments, the kit comprises two soft tissue-stretching straps 60 and at least two strengthening straps 64, each strengthening strap 64 comprising an elastic resistance member 68 of unique elastic resistance.

In some embodiments, the kit comprises a plurality of tethers 66 which are interchangeable with a plurality of elastic resistance members 68. It is envisioned that the elastic resistance members 68 possess certain elastic resistance characteristics which may be varied by altering the material or thickness of the elastic resistance member 68. In some embodiments, the kit comprises a plurality of elastic resistance members 68, each having a unique elastic resistance.

The components of such kit may be held in a carrying bag sized to fit all the components. In various embodiments, the bag may be comprised of a fabric or leather material. In further embodiments, the carrying bag is sealable through means understood in the art (e.g., via drawstring, zipper, hook and loop, or snap buttons). In some embodiments, the kit may include further modular components that may take the place of brace 20, digit immobilizer 40, the at least one strap, or upper arm support 80. The kit may further or alternatively include packaging having one or more compartments. The kit may also include adhesives, hook and loop strips, instructions and/or other procedural supplies for using device 10.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A therapeutic device for the manipulative treatment of soft tissue of the human body, the device comprising:
a brace comprising a curved body that partially or completely encompasses a portion of an arm; and
a digit immobilizer comprising at least one plate to receive a hand in a flat position,
wherein the brace and the digit immobilizer are in communication with one another through at least one strap extending from the curved body of the brace to one of the plates of the digit immobilizer.

2. The therapeutic device according to claim 1, wherein the curved body of the brace comprises a C-shaped cross-section.

3. The therapeutic device according to claim 1 or 2, wherein the curved body of the brace comprises a gap at opposing ends of the curved body.

4. The therapeutic device according to claim 3, wherein the brace comprises at least one fastener spanning the gap to secure the brace firmly in place on a user's arm.

5. The therapeutic device according to any of the preceding claims, wherein the brace comprises a cushion member at one or both of the proximal and distal ends of the brace.

6. The therapeutic device according to any of the preceding claims, wherein the at least one strap extends from a distal end of the curved body of the brace to one of the plates of the digit immobilizer.

7. The therapeutic device according to any of the preceding claims, comprising a pair of straps extending respectively from a distal end of the curved body of the brace to one of the plates of the digit immobilizer.

8. The therapeutic device according to claim 7, wherein the first strap extends from a distal end of the curved body on a lateral-facing portion of the brace to a lateral plate, and the second strap extends from a distal end of the curved body on a medial-facing portion of the brace to a medial plate.

9. The therapeutic device according to any of the preceding claims, wherein the at least one strap is a soft tissue-stretching strap or a strengthening strap.

10. The therapeutic device according to any of the preceding claims, wherein the strap is a soft tissue-stretching strap having an adjustable length.

11. The therapeutic device according to any of the preceding claims, wherein the static soft tissue-stretching strap is substantially or completely non-elastic.

12. The therapeutic device according to any of the preceding claims, wherein the static soft tissue-stretching strap has a fixable length such that when in place extending between the brace and the digit immobilizer, one of extension or flexion of the wrist is limited.

13. The therapeutic device according to any of claims 1-9, wherein the strap is a strengthening strap comprising an elastic resistance member.

14. The therapeutic device according to claim 13, wherein the strengthening strap comprises an elastic resistance member having an elastic resistance such that when the strengthening strap is in place extending between the brace and the digit immobilizer, resistance force is placed on the wrist against one of extension or flexion.

15. The therapeutic device according to any of the preceding claims, wherein the digit immobilizer comprises a medial plate disposable with or adjacent to the palm of a hand and a lateral plate disposable with or adjacent to the back of the hand.

16. The therapeutic device according to any of the preceding claims, wherein the digit immobilizer comprises a glove in which the plate(s) may be received to fit directly to a user's hand.

17. The therapeutic device according to claim 16, wherein the glove medially receives a first plate to position against the palm, and laterally receives a second plate to position against the back of the hand.

18. The therapeutic device according to any of the preceding claims, further comprising an upper arm support having a curved body.

19. The therapeutic device according to claim 18, further comprising at least one connecting member to secure a distal end of the upper arm support to a proximal end of the brace.

20. The therapeutic device according to any of the preceding claims, wherein the device is useful in the treatment of tendons, ligaments, or muscles in the arm.

21. The therapeutic device according to any of the preceding claims, wherein the device is useful in the treatment of carpal tunnel syndrome, tendonitis, epicondylitis (i.e., tennis elbow), and hand-arm vibration syndrome (e.g., treatment of lateral epicondylitis).

22. A therapeutic method for rehabilitating soft tissue of the human body, the method comprising the following steps:
providing a therapeutic device comprising:
a brace comprising a curved body that partially or completely encompasses a portion of an arm; and
a digit immobilizer comprising at least one plate to receive a hand in a flat position,
wherein the brace and the digit immobilizer are in communication with one another through at least one strap extending from the curved body of the brace to one of the plates of the digit immobilizer;
placing the digit immobilizer on a user's hand and placing the brace on a portion of the user's arm; and
attaching the at least one strap between the brace and the digit immobilizer for a desired therapeutic effect.

23. The method according to claim 22, wherein the therapeutic device is a device according to any of claims 1-21.

24. The method according to claim 22 or 23, wherein the desired therapeutic effect is stretching or strengthening of soft tissue.

25. The method according to any of claims 22-24, wherein the placing step further comprises looping bands around the plates of the digit immobilizer and fasteners on the brace are tightened such that the digit immobilizer is firmly engaged with the user's hand and the brace is firmly engaged with the user's arm.

26. The method according to any of claims 22-25, the desired therapeutic effect is stretching of the lateral forearm muscle group of a user's forearm, and wherein attaching the strap between the brace and the digit immobilizer comprises attachment of a first end of the strap to an outer surface of a medial plate and a second end of the strap to a medial engagement loop on the brace; or wherein the desired therapeutic effect is stretching of a medial forearm muscle group of a user's forearm, and wherein attaching the strap between the brace and the digit immobilizer comprises attachment of a first end of the strap to an outer surface of a lateral plate and a second end of the strap to a lateral engagement loop on the brace.

27. The method according to claim 26, further comprising the step of adjusting the length of the strap to lock the wrist in a forearm extensor stretching position or a forearm flexor stretching position.

28. The method according to any of claims 22-25, wherein the desired therapeutic effect is strengthening of muscle, and the strap is a strengthening strap.

29. The method according to claim 28, wherein the strengthening strap comprises an elastic resistance member with sufficient elastic resistance such that when the strengthening strap is in place extending between the brace and the digit immobilizer, resistance force is placed on the wrist against one of extension or flexion.

30. The method according to any of claims 22-29, wherein further comprising the step of placing the upper arm support on a portion of the arm proximal to the brace, and further tightening fasteners on the upper arm support such that the upper arm support is firmly engaged with the user's upper forearm and optionally the elbow.

31. A kit for a therapeutic device for the manipulative treatment of soft tissue of the human body, the kit comprising:
a brace comprising a curved body that partially or completely encompasses a portion of an arm;
a digit immobilizer comprising at least one plate to receive a hand in a flat position; and
a strap attachable at one end to the curved body of the brace and at the other end to one of the plates of the digit immobilizer.
